(19) United States
(12) Patent Application Publication
(10) Pub. No.: US 2022/0172355 A1
(43) Pub. Date: Jun. 2, 2022

(54) CYTOLOGICAL ANALYSIS OF NUCLEAR NEAT-1 EXPRESSION FOR DETECTION OF CHOLANGIOCARCINOMA

(71) Applicant: Mayo Foundation for Medical Education and Research, Rochester, MN (US)

(72) Inventor: Tushar C. Patel, Ponte Vedra Beach, FL (US)

(21) Appl. No.: 17/539,468

(22) Filed: Dec. 1, 2021

Related U.S. Application Data
(60) Provisional application No. 63/120,496, filed on Dec. 2, 2020.

(51) Int. Cl. G06T 7/00; G06V 20/69; G06V 10/56; G06V 10/82; C12Q 1/6841; C12Q 1/6886

(52) U.S. Cl. CPC G06T 7/0012; G06V 20/695; G06V 10/56; G06V 10/82; C12Q 2600/158; C12Q 1/6886; G06T 2207/30024; G06T 2207/10024; G06T 2207/20084; C12Q 1/6841

(57) ABSTRACT

A method for detecting malignancy in a biliary cytology sample. In situ hybridization is performed on the biliary cytology sample to express nuclear paraspeckle assembly transcript 1 (NEAT-1). The hybridized biliary cytology sample is imaged, and the NEAT-1 expressions are identified. A plurality of features of the identified NEAT-1 expressions are detected. The detected features of the NEAT-1 expressions are processed by a processor configured with algorithm criteria defined by a trained neural network and based on the plurality of features to provide predictions of malignancy in the sample.

### CROSS-REFERENCE TO RELATED APPLICATION

This application claims the benefit of Provisional Application No. 63/120,496, filed Dec. 2, 2020, which is incorporated herein by reference in its entirety for all purposes.

### FIELD

This disclosure relates generally to methods and systems for the detection of certain cancers such as cholangiocarcinoma—a malignancy affecting the epithelial lining of the biliary tract. In particular, disclosed embodiments include the use of image processing to detect and analyze certain features of nuclear paraspeckle assembly transcript 1 (NEAT-1) expressions in biliary cells following RNA in situ hybridization.

### BACKGROUND

Cholangiocarcinoma is a malignancy or cancer affecting the epithelial lining of the biliary tract. Known approaches for diagnosing cholangiocarcinoma include cytological analysis of morphological and cellular features of tissue collected from regions of interest. Tissue biopsy may be used to collect the samples for analysis. However, it may be difficult to obtain adequate tissue for diagnosis from areas of the bile ducts or from biliary tract strictures because of their fibrotic nature. In addition, tissue biopsies may be less commonly performed due to risks of complications such as scarring with stricture formation, hemorrhage or bile leaks.

Tissue cell samples may be collected from the biliary tract by brush cytology during biliary tract endoscopy. The cells are stained by hematoxylin and eosin (H&E) or Papanicolaou (Pap) stain. The stained cells are then examined by a cytopathologist using microscopy to determine whether or not malignancy is present. This approach has a relatively low complication rate, is available and relatively easy to perform, and may enable sampling of the entire extrahepatic biliary tract. However, the diagnostic accuracy of the approach may be relatively low. Although this approach may have relatively high specificity, it may have very low sensitivity.

Fluorescence in situ hybridization (FISH) is another known approach for diagnosing cholangiocarcinoma, and is sometimes used when other cytological analysis is negative or inconclusive. FISH may provide increased sensitivity while preserving the specificity of cytological analysis.

In addition to the risks of complications, these and other known technologies may provide inconclusive diagnoses in significant numbers of samples. There remains a continuing need for improved methods and systems for the accurate diagnosis of biliary cancers.

### SUMMARY

Methods and systems for the accurate detection of malignancy in biliary cytology are disclosed. Embodiments of the method are based on the analysis of nuclear morphological changes associated with malignancy using algorithms to optimize detection of malignancy based on imaging features after RNA in situ hybridization is performed for expression of a nuclear RNA. The nuclear RNA expressions may be nuclear paraspeckle assembly transcript 1 (NEAT-1).

One example is a method for detecting malignancy in a biliary cytology sample. Embodiments of the method may comprise: performing in situ hybridization on the biliary cytology sample to express nuclear paraspeckle assembly transcript 1 (NEAT-1) in the biliary cytology sample; imaging at least portions of the in situ hybridized biliary cytology sample and identifying the NEAT-1 expressions; detecting a plurality of features of the identified NEAT-1 expressions; and processing the detected features of the NEAT-1 expressions by one or more processors configured with algorithm criteria based on the plurality of features to provide predictions of malignancy in the sample. In embodiments, the plurality of features may comprise two or more features from the set including (1) maximum intensity, (2) colocation, (3) minimum diameter, (4) a first component of a first color space (optionally the in-phase component I of the YIQ color space), (5) average intensity, (6) a first component of a second color space (optionally intensity blue of the rgb color space), (7) centroid, (8) a first component of a third color space (optionally the b component of the Lab color space), and (9) saturation.

In embodiments of any or all of the above methods, the plurality of features may comprise at least: the maximum intensity, wherein the maximum intensity is within a range of maximum intensities including one or more of (1) a first maximum intensity threshold, (2) a second maximum intensity threshold that is less than the first maximum intensity threshold, (3) a third maximum intensity threshold that is less than the second maximum intensity threshold, (4) a fourth maximum intensity threshold that is less than the third maximum intensity threshold, (5) a fifth maximum intensity threshold that is less than the fourth maximum intensity threshold, (6) a sixth maximum intensity threshold that is less than the fifth maximum intensity threshold, (7) a seventh maximum intensity threshold that is less than the sixth maximum intensity threshold, and (8) an eighth maximum intensity threshold that is less than the seventh maximum intensity threshold; and the colocation. In such embodiments, the algorithm criteria may predict malignancy when: the maximum intensity is less than a first maximum intensity threshold; and the colocation is greater than or equal to a colocation threshold.

In embodiments of any or all of the above methods, the plurality of features may further comprise: the minimum diameter; and the first component of the first color space, wherein the first component of the first color space is within a range of first components of the first color space including one or more of (1) a first first color space first component threshold, and (2) a second first color space first component threshold that is greater than the first first color space first component threshold. In such embodiments, the algorithm criteria may predict malignancy when: the maximum intensity is less than the first maximum intensity threshold; the colocation is less than the colocation threshold; the minimum diameter is greater than or equal to a minimum diameter threshold; and the first component of the first color space is less than a first first color space first component threshold.

In embodiments of any or all of the above methods, the plurality of features may further comprise the mean intensity. In such embodiments, the algorithm criteria may predict malignancy when: the maximum intensity is less than the first maximum intensity threshold; the colocation is less than the colocation threshold; the minimum diameter is greater than or equal to the minimum diameter threshold; the first component of the first color space is greater than or equal to the first first color space first component threshold; and the mean intensity is less than a mean intensity threshold.

In embodiments of any or all of the above methods, the algorithm may predict malignancy when: the maximum intensity is less than the first maximum intensity threshold and greater than or equal to the third maximum intensity threshold; the colocation is less than the colocation threshold; the minimum diameter is greater than or equal to the minimum diameter threshold; the first component of the first color space is greater than or equal to the first first color space first component threshold; and the mean intensity is greater than or equal to the mean intensity threshold.

In embodiments of any or all of the above methods, the algorithm may predict malignancy when: the maximum intensity is less than the eighth maximum intensity threshold; the colocation is less than the colocation threshold; and the minimum diameter is less than the minimum diameter threshold.

In embodiments of any or all of the above methods, the plurality of features may further comprise the first component of the second color space. In such embodiments the algorithm criteria may predict malignancy when: the maximum intensity is less than the first maximum intensity threshold and greater than or equal to the fourth maximum intensity threshold; the colocation is less than the colocation threshold; the minimum diameter is less than the minimum diameter threshold and the intensity of the first component of the second color space is less than a second color space first component threshold.

In embodiments of any or all of the above methods, the plurality of features may further comprise the centroid. In such embodiments, the algorithm criteria may predict malignancy when: the maximum intensity is less than the fourth maximum intensity threshold and greater than or equal to the seventh maximum intensity threshold; the colocation is less than the colocation threshold; the minimum diameter is less than the minimum diameter threshold; the first component of the second color space is less than the second color space first component threshold; and the centroid is greater than or equal to a centroid threshold.

In embodiments of any or all of the above methods, the plurality of features may further comprise the first component of the third color space. In such embodiments, the algorithm criteria may predicts malignancy when: the maximum intensity is less than the fourth maximum intensity threshold and greater than or equal to the seventh maximum intensity threshold; the colocation is less than the colocation threshold; the minimum diameter is less than the minimum diameter threshold; the first component of the second color space is less than the second color space first component threshold; the centroid is less than the centroid threshold; and the first component of the third color space is less than a third color space first component threshold.

In embodiments of any or all of the above methods, the plurality of features may further comprise the saturation. In such embodiments, the algorithm criteria may predict malignancy when: the maximum intensity is less than the second maximum intensity threshold and greater than or equal to the fifth maximum intensity threshold; the colocation is less than the colocation threshold; the minimum diameter is less than the minimum diameter threshold; the first component of the second color space is greater than or equal to the second color space first component threshold; and the saturation is greater than or equal to a saturation threshold.

In embodiments of any or all of the above methods, the algorithm may predict malignancy when: the maximum intensity is less than the sixth maximum intensity threshold and greater than or equal to the seventh maximum intensity threshold; the colocation is less than the colocation threshold; the minimum diameter is less than the minimum diameter threshold; the first component of the first color space is less than a second first color space first component threshold that is greater than the than the first first color space first component threshold; and the first component of the second color space is greater than or equal to the second color space first component threshold.

In embodiments of any or all of the above methods, processing the detected features may include processing the detected features by one or more processors executing instructions of a trained neural network defining the algorithm criteria.

Another example is a computing device configured to execute instructions defining the algorithm criteria of any or all of the above methods. Yet another example is a computer-readable medium including stored instructions to cause one or more processors to carry out the algorithm criteria of any or all of the above methods.

### BRIEF DESCRIPTION OF THE DRAWINGS

FIG. 1 is a diagrammatic illustration of a method for diagnosing cancer such as cholangiocarcinoma, in accordance with embodiments.

FIG. 2 is a diagrammatic illustration of laboratory equipment that can be used in connection with the method of FIG. 1, in accordance with embodiments.

FIGS. 3A and 3B are exemplary images of tissue samples at stages during the performance of the method of FIG. 1, in accordance with embodiments.

FIG. 4 is a list of NEAT-1 features that may be detected and used in connection with the method of FIG. 1, in accordance with embodiments.

FIG. 5 is diagrammatic illustration of components of the computer system shown in FIG. 2, in accordance with embodiments.

FIG. 6A is a list of detected NEAT-1 features that may be used in connection with the method of FIG. 1, in accordance with embodiments.

FIG. 6B is a listing of algorithm criteria based on the features of FIG. 6A that may be used in connection with the method of claim 1, in accordance with embodiments.

FIG. 7 is a decision tree representation of the algorithm criteria shown in FIG. 6B.

### DETAILED DESCRIPTION

Methods for detecting biliary tract malignancies in accordance with this disclosure include performing in situ hybridization of biliary tract tissues to cause the expression of nuclear paraspeckle assembly transcript 1 (NEAT-1) in the tissues. NEAT-1 is a nuclear long non-coding RNA that is upregulated and plays an oncogenic role in many types of solid tumors. NEAT-1 can function as an important structural component of a nuclear domain known as paraspeckle, which participates in the regulation of gene expression through the nuclear retention of proteins and RNAs. NEAT-1 is an important nuclear component and its knockdown results in the integration of paraspeckles. Paraspeckles have been shown to participate in the regulation of gene expression by keeping mRNAs in the nucleus for editing. NEAT-1 is transcribed from the familial tumor syndrome multiple endocrine neoplasia type 1 locus, located on chromosome 11. The Neat1 gene encodes two transcriptional variants, namely NEAT-1 and NEAT-2. The two variants, both localized to nuclear paraspeckles, share the same promoter with different 3′-end processing mechanisms. Analysis of certain features of the NEAT-1 expressions may provide an effective and accurate basis to predict malignancy or cancer, such as cholangiocarcinoma, that may be present in the tissues.

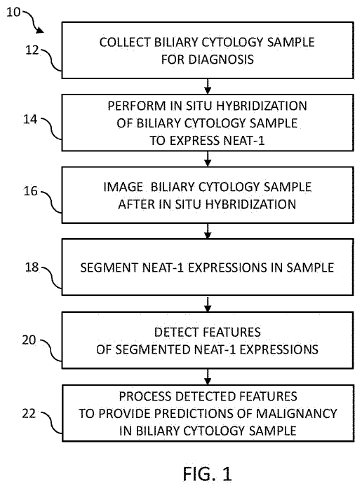

FIG. 1 is a diagrammatic illustration of a method 10 for detecting malignancy in biliary cytology samples in accordance with embodiments. The method includes detecting nuclear morphological changes related to alterations in nuclear NEAT-1 expression in malignant cells. As shown, the method includes: (1) collecting a biliary cytology sample (step 12), (2) performing in situ hybridization of the sample to cause the expression of nuclear paraspeckle assembly transcript 1 (NEAT-1) in the sample (step 14), (3) imaging the sample after in situ hybridization has been performed, and generating one or more electronic images of the sample including some or all of any NEAT-1 expressions in the sample (step 16), (4) processing the electronic images to segment one or more of any NEAT-1 expressions in the images (step 18), (5) processing the images of the segmented NEAT-1 expressions to detect a plurality of features of the NEAT-1 expressions (step 20), and (6) processing the detected features of the NEAT-1 expressions using an algorithm based on the plurality of features to provide predictions of malignancy in the biliary cytology sample (step 22).

In connection with step 12, biliary cytology samples of tissues for analysis and diagnosis can, for example, be collected from a patient's biliary tract using conventional brush cytology or otherwise known techniques. In embodiments, the cytology samples may be collected from areas of the bile ducts or from biliary tract strictures. In other embodiments the cytology samples may collected by tissue biopsy. Biliary cytology sample collection techniques of these types are disclosed, for example, in the following references, which are incorporated herein by reference in their entireties and for all purposes: (1) Peter V. Draganov et al., Diagnostic accuracy of conventional and cholangioscopy-guided sampling of indeterminate biliary lesions at the time of ERCP: a prospective, long-term follow-up study, Gastrointestinal Endoscopy, vol. 75, no. 2, pp. 347-353 (2012), and (2) R. Temino Lopez-Jurado et al., Rev. Esp. Enferm. Dig., 101 (6), pp. 385-394 (2009). Cell preparations of the collected tissue samples may be prepared, for example on glass slide, using conventional or otherwise known techniques.

As additional examples, biliary cytology samples used in connection with the development of methods described herein were collected by brush cytology protocols using a cytology brush including bristles made of nylon fibers that branch off a thin metal shaft and that run lengthwise within a protective plastic sheath. The cytology brush was passed through an accessory channel of an endoscope and used to sample the mucosa, by rubbing the brush back and forth several times along the surface of a lesion or stricture. The brush was then pulled back into the sheath and removed from the endoscope. The brush was subsequently pushed out of the sheath to expose the bristles, and the exposed bristles were smeared against a glass slide to deposit the tissue sample on the slide. The glass slide with the tissue sample was then immediately submerged or sprayed with fixative.

In connection with step 14, conventional or otherwise known technologies and techniques can be used to process and perform the in situ hybridization of the sample. Suitable in situ hybridization techniques for expressing NEAT-1 are disclosed, for example in the following references which are incorporated herein by reference in their entireties and for all purposes: S. Nakagawa et al., Paraspeckles are subpopulation-specific nuclear bodies that are not essential in mice. J Cell Biol (2011) 193 (1): 31-39; Y. Nishimoto et al., The long non-coding RNA nuclear-enriched abundant transcript 1_2 induces paraspeckle formation in the motor neuron during the early phase of amyotrophic lateral sclerosis, Mol Brain. 2013, 6: 31. In connection with the development of the methods described herein, for example, processing including RNA in situ hybridization was performed using RNAscope Probe Hs-NEAT1-long, available from Advanced Cell Diagnostics, Inc. of Newark, Calif. (catalog no. 41151). This probe is characterized Accession No. NC 00011.9 and Target Region 4120-5238. Other embodiments may use other suitable probes (e.g., Stellaris FISH probes, Human NEAT1 5′ Segment with Quasar 570 dye (catalog no. SMF-2036-1)).

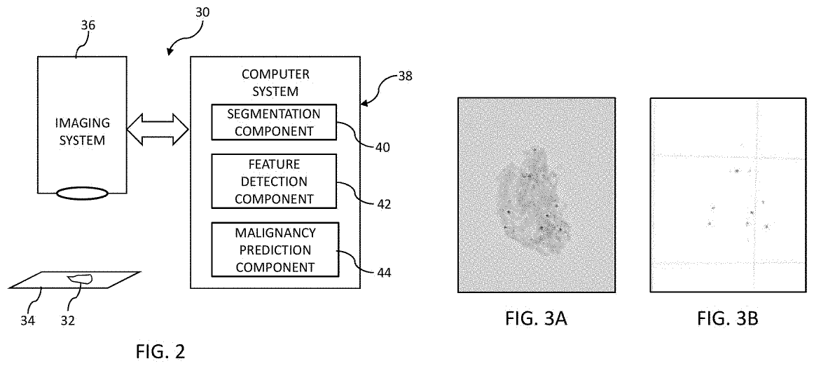

FIG. 2 is a diagrammatic illustration of laboratory equipment 30 that can be used to perform the imaging and processing steps 16, 18, 20 and 22 on a biliary cytology sample 32. The sample 32 has been processed in the manner described above to provide NEAT-1 expressions, and is shown on a slide 34 for purposes of example. In embodiments, the biliary cytology sample 32 is processed in a manner that enables conventional fluoroscopy microscopy and image processing during the steps 16, 18 and 20. As shown, laboratory equipment 30 includes an imaging system 36 and a computer system 38. Imaging system 36 may be any suitable conventional or otherwise known imaging system. As an example, an Aperio AT2 Scanner from Leica Biosystems imaging system may be used in embodiments. Imaging system 36 images the nuclear morphology and subcellular nuclear RNA expressions in the sample 32, and generates image data representative of the sample images. FIG. 3A is an exemplary image of a cell of a processed biliary cytology sample 32 including NEAT-1 expressions (visible as the relatively small dark areas at locations throughout the cell).

Computer system 38 is coupled to the imaging system 36 and includes a segmentation component 40, a feature detection component 42 and a malignancy prediction component 44. Segmentation component 40 processes the images of the biliary cytology samples received from the imaging system 36 and segments the one or more NEAT-1 expressions that may be present in the images from surrounding tissues. In embodiments, the segmentation component segments the NEAT-1 expressions at a nuclear, subcellular level. The functionality of segmentation component 40 can be provided by computer system 38 using any suitable conventional or otherwise known image processing software. As an example, Arivis (from Arivis), Imaris (from Oxford Instruments), or Celleste (from ThermoFisher Scientific) image processing software may be used in embodiments. FIG. 3B is an exemplary image of the NEAT-1 expressions segmented from the image of FIG. 3A.

Feature detection component 42 processes the images of the segmented NEAT-1 expressions and generates data characteristic of a plurality of characteristics or features of the expressions. The functionality of the feature detection component 42 can be provided by computer system 38 using any suitable conventional or otherwise known image processing software. In embodiments, for example, the functionality of the feature detection component 42 is provided by the same image processing software used to provide the functionality of the segmentation component 40 and described above. As described above, embodiments of the method 10 performed by laboratory equipment 30 utilize fluorescence microscopy. Other embodiments utilize additional and/or alternative technologies, such as brightfield processing, confocal, multi-proton, or super-resolution microscopy and imaging approaches.

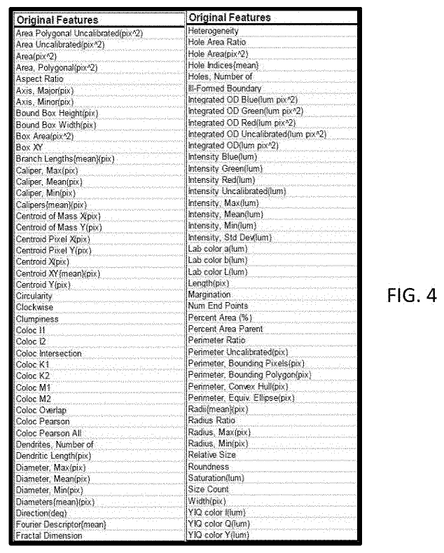

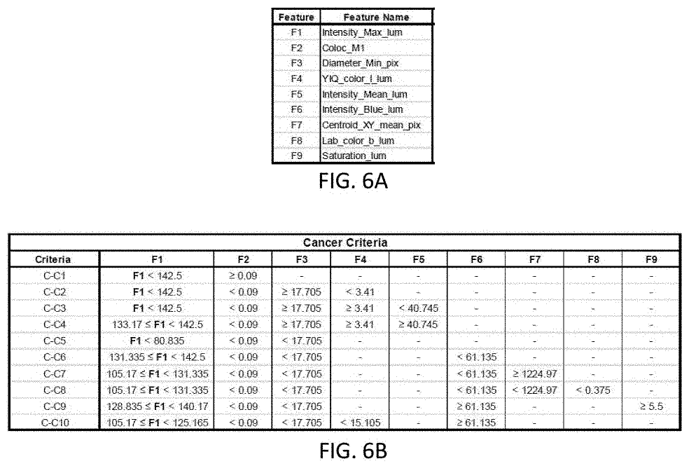

Malignancy prediction component 44 processes the features detected by the feature detection component 42 using a prediction algorithm based on the detected features, and provides predictions of malignancy in the biliary cytology sample. The predictions provided by the malignancy prediction component 44 are based on the results provided by the algorithm in response to the detected features. Malignancy prediction component 44 processes two or more detected features by the prediction algorithm to provide the malignancy predictions. FIG. 4 is a table listing examples of fluorescence microscopy features of NEAT-1 expressions that may be detected by the feature detection component 42 and processed by the malignancy prediction component 44 using a prediction algorithm to provide the malignancy predictions. In embodiments described in greater detail below, the method 10 detects at step 42 and processes at step 44 the nine features of NEAT-1 expressions described below in Table 1 (and listed in FIG. 6A) for purposes of predicting malignancies in the biliary cytology samples.

TABLE 1

| Feature No. | Feature Name | Description |
|---|---|---|
| F1 | Intensity_Max_lum | Maximum luminescent intensity (i.e., |
| | | Maximum Intensity of all the segmented |
| | | objects) |
| F2 | Coloc_M1 | Colocation of portion of intensity in the |
| | | segmented region within a signal channel that |
| | | coincides with some (any) intensity in any of |
| | | the other channels |
| F3 | Diameter_Min_pix | Minimum diameter (expressed in pixels) (i.e., |
| | | Minimum Diameter observed among all of the |
| | | segmented objects |
| F4 | YIQ_col or_I_lum | Luminescent intensity of the in-phase |
| | | component I in the YIQ color space (i.e., a First |
| | | Component of a First Color Space), averaged |
| | | from all of the segmented objects |
| F5 | Intensity_Mean_lum | Mean luminescent intensity of all the identified |
| | | expressions from all of the segmented objects |
| | | (i.e., Average Intensity) |
| F6 | Intensity_Blue_lum | Luminescent intensity of the blue component in |
| | | the RGB color space (i.e., a First Component of |
| | | a Second Color Space), averaged from all of the |
| | | segmented objects |
| F7 | Centroid_XY_mean_pix | Centroid, averaged from all of the segmented |
| | | objects (expressed in pixels) (i.e., Average |
| | | Centroid) |
| F8 | Lab_color_b_lum | Luminescent intensity of the b component in |
| | | the Lab color space (i.e., a First Component of a |
| | | Third Color Space), averaged from all of the |
| | | segmented objects |
| F9 | Saturation_lum | Luminescent saturation (i.e., Saturation) |
| | | averaged from all of the segmented objects |

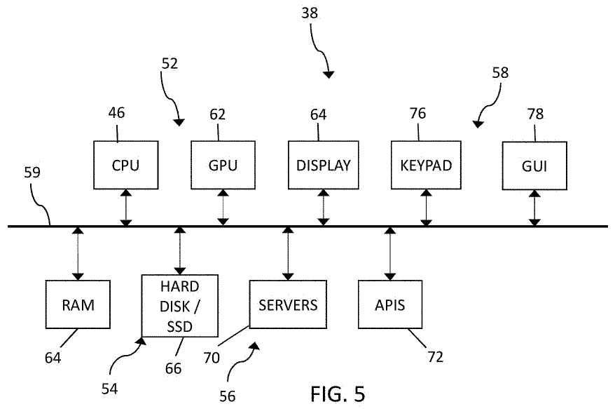

FIG. 5 is a diagrammatic illustration of an exemplary computer system 38 that may be used to implement the segmentation component 40, feature detection component 42 and malignancy prediction component 44 in accordance with embodiments. The illustrated embodiments of computer system 38 comprise processing components 52, storage components 54, network interface components 56 and user interface components 58 coupled by a system network or bus 59. Processing components 52 may, for example, include central processing unit (CPU) 60 and graphics processing unit (GPU) 62, and provide the processing functionality segmentation component 40, feature detection component 42 and malignancy prediction component 44. The storage components 54 may include RAM memory 64 and hard disk/SSD memory 66, and provide the storage functionality of the segmentation component 40, feature detection component 42 and malignancy prediction component 44. For example, operating system software used by the processing components 52 and one or more image processing application software packages used by the segmentation component 40 and/or feature detection component 42 to perform the segmentation step 18 and/or feature detection step 20 to implement methods described herein may be stored by the storage components 54. Software programs used by the malignancy prediction component 44 and configured with algorithms based on the detected features to perform the processing step 22 may also be stored by the storage components 54. In embodiments, the network interface components may include one or more web servers 70 and one or more application programming interfaces (APIs) 72 (e.g., for coupling the computer system 38 to the imaging system 36. Examples of user interface components 58 include display 74, keypad 76 and graphical user interface (GUI) 78. Embodiments of computer system 38 may include other conventional or otherwise known components to implement malignancy prediction methods such as 10 in accordance with embodiments described herein.

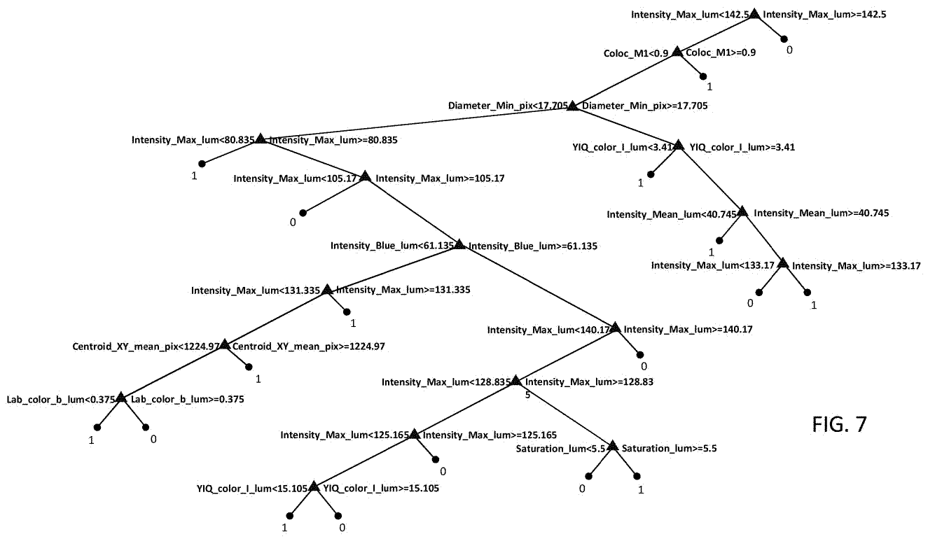

FIG. 6A is an illustration of a table corresponding to Table 1 above listing the nine features F1-F9 that are used in embodiments of the malignancy prediction algorithm described herein. FIG. 6B is a table listing a set of ten criteria C-C1-C-C10, each based on two or more of the features F1-F9, that describe or define components of embodiments of the malignancy prediction algorithm. FIG. 7 is a decision tree graphically illustrating the criteria C-C1-C-C10 shown in the table of FIG. 6B. Each of the criteria C-C1-C-C10 independently defines a feature-based equation having a binary output, were one output represents a normal sample condition (i.e., predicting no malignancy), and the other output represents a malignant sample condition (i.e., predicting malignancy or cancer). In the convention used by the criteria C-C1-C-C10 described in FIGS. 6B and 7, an output of “0” (i.e., the respective criteria is false) represents a prediction of a normal sample condition, and an output of “1” (i.e., the respective criteria is true) represents a prediction of a malignant sample. Embodiments of method 10 may make use or any one or more of the criteria C-C1-C-C10 as a basis for malignancy predictions. In embodiments, all criteria C-C1-C-C10 and associated features F1-F9 are applied to the processed and imaged biliary cytology samples for purposes of providing malignancy predictions. By such embodiments, the method 10 predicts a normal condition for a sample if all criteria C-C1-C-C10 provide “0” outputs, and predicts a malignant condition for the sample if any one or more of the criteria C-C1-C-C10 provide a “1” output.

Criteria C-C1-C-C10 describe equations comparing the detected values of the features to threshold values (e.g., predetermined values) for the associated features. In embodiments, two or more criteria based on the same features may use different threshold values associated with a given feature. In the illustrated embodiments, for example, criteria C-C2-C-C4 use a first threshold value for feature F4 (e.g., 3.41 for YIQ_color_I_lum, i.e., a first component of a first color space), while criteria C-C10 uses a second threshold value for feature F4 (e.g., 15.105). In the illustrated embodiments, criteria C-C1-C-C10 use one or more of eight different threshold values for feature F1 (e.g., maximum intensity). The threshold values for feature F1 used by criteria C-C1-C-C10 extend over a range of maximum intensity values. For purposes of description, the threshold values for feature F1 can be characterized as decreasing in value sequentially from a first threshold having a greatest value (e.g., 142.5 in the illustrated embodiments), through second, third, fourth, fifth, sixth and seventh thresholds, to an eighth threshold having the lowest value (e.g., 80.835 in the illustrated embodiments). Criteria C-C1-C-C10 in accordance with embodiments, and corresponding to the embodiments shown in FIGS. 6B and 7, are described below in Table 2. It is to be understood that although particular threshold values as listed, other embodiments my use other threshold values.

TABLE 2

| Criteria | |
|---|---|
| No. | Feature-Based Criteria Equations Predicting Malignancy |
| C-C1 | F1 < 142.5 (i.e., a first maximum intensity threshold) AND F2 ≥ 0.09 (i.e., a |
| | colocation threshold) |
| C-C2 | F1 < 142.5 AND F2 < 0.09 AND F3 ≥ 17.705 (i.e., a minimum diameter threshold) |
| | AND F4 < 3.41 (i.e., a first, first color space first component threshold) |
| C-C3 | F1 < 142.5 AND F2 < 0.09 AND F3 ≥ 17.705 AND F4 ≥ 3.41 AND F5 < 40.745 |
| | (i.e., a mean intensity threshold) |
| C-C4 | 133.17 (i.e., a third maximum intensity threshold) < F1 < 142.5 AND F2 < 0.09 |
| | AND F3 ≥ 17.705 AND F4 ≥ 3.41 AND F5 ≥ 40.745 |
| C-C5 | F1 < 80.835 (i.e., an eighth maximum intensity threshold) AND F2 < 0.09 AND |
| | F3 < 17.705 |
| C-C6 | 131.335 (i.e., a fourth maximum intensity threshold) ≤ F1 < 142.5 AND F2 < 0.09 |
| | AND F3 < 17.705 AND F6 < 61.135 (i.e., a second color space first component |
| | threshold |
| C-C7 | 105.17 (i.e., a seventh maximum intensity threshold) ≤ F1 < 131.335 AND F2 < |
| | 0.09 AND F3 < 17.705 AND F6 < 61.135 AND F7 ≥ 1224.97 (i.e., an average |
| | centroid threshold) |
| C-C8 | 105.17 ≤ F1 < 131.335 AND F2 < 0.09 AND F3 < 17.705 AND F6 < 61.135 AND |
| | F7 < 1224.97 AND F8 < 0.375 (i.e., a third color space first component threshold) |
| C-C9 | 128.835 (i.e., a fifth maximum intensity threshold) ≤ F1 < 140.1 (i.e., a second |
| | maximum intensity threshold) AND F2 < 0.09 AND F3 < 17.705 AND F6 ≥ |
| | 61.135 AND F9 ≥ 5.5 (i.e., a saturation threshold) |
| C-C10 | 105.17 ≤ F1 < 125.165 (i.e., a sixth maximum intensity threshold) AND F2 < 0.09 |
| | AND F3 < 17.705 AND F4 < 15.105 (i.e., a second, first color space first |
| | component threshold) AND F6 ≥ 61.135 |

Algorithms criteria for use in connection with embodiments of method 10 may be developed as a decision tree or as neural networks (e.g., by machine learning methodologies). Embodiments of the algorithm represented by one or more of the criteria C-C1-C-C10 and associated features F1-F9 were developed using trained neural network methodologies using biliary cytology samples determined by other methods (e.g., those described above in the Background section) to be benign or malignant for cholangiocarcinoma. Conventional or otherwise known analytics platforms such as the Konstanz Information Miner (KNIME) analytics platform available from KNIME AG, Zurich, Switzerland, may be used for the development of such algorithms. Embodiments of method 10 may be used with algorithm criteria different than those of criteria C-C1-C-C10. Algorithms for use with method 10 may also be developed by other methodologies, as untrained neural networks.

Methods for detecting malignancy in biliary cytology in accordance with embodiments described herein have demonstrated high degrees of accuracy with high sensitivity and high specificity. High precision and high F1 scores were also demonstrated. In embodiments, the methods may be used when conventional cytology or FISH approaches are inconclusive. The analysis of nuclear morphological changes associated with malignancy may improves diagnostic utility of cytology for the detection of biliary tract cancer in patients that have biliary tract strictures.

It is to be understood that the above description is intended to be illustrative, and not restrictive. Many other embodiments will be apparent to those of skill in the art upon reading and understanding the above description. It is contemplated that features described in association with one embodiment are optionally employed in addition or as an alternative to features described in or associated with another embodiment. The scope of the invention should, therefore, be determined with reference to the appended claims, along with the full scope of equivalents to which such claims are entitled.

1. A method for detecting malignancy in a biliary cytology sample, comprising:
performing in situ hybridization on the biliary cytology sample to express nuclear paraspeckle assembly transcript 1 (NEAT-1) in the biliary cytology sample;
imaging at least portions of the in situ hybridized biliary cytology sample and identifying the NEAT-1 expressions;
detecting a plurality of features of the identified NEAT-1 expressions; and
processing the detected features of the NEAT-1 expressions by one or more processors configured with algorithm criteria based on the plurality of features to provide predictions of malignancy in the sample.

2. The method of claim 1 wherein the plurality of features comprises two or more features from the set including (1) maximum intensity, (2) colocation, (3) minimum diameter, (4) a first component of a first color space (optionally the in-phase component I of the YIQ color space), (5) average intensity, (6) a first component of a second color space (optionally intensity blue of the rgb color space), (7) centroid, (8) a first component of a third color space (optionally the b component of the Lab color space), and (9) saturation.

3. The method of claim 2 wherein the plurality of features comprises at least:
the maximum intensity, wherein the maximum intensity is within a range of maximum intensities including one or more of (1) a first maximum intensity threshold, (2) a second maximum intensity threshold that is less than the first maximum intensity threshold, (3) a third maximum intensity threshold that is less than the second maximum intensity threshold, (4) a fourth maximum intensity threshold that is less than the third maximum intensity threshold, (5) a fifth maximum intensity threshold that is less than the fourth maximum intensity threshold, (6) a sixth maximum intensity threshold that is less than the fifth maximum intensity threshold, (7) a seventh maximum intensity threshold that is less than the sixth maximum intensity threshold, and (8) an eighth maximum intensity threshold that is less than the seventh maximum intensity threshold; and
the colocation.

4. The method of claim 3 wherein the algorithm criteria predicts malignancy when:
the maximum intensity is less than a first maximum intensity threshold; and
the colocation is greater than or equal to a colocation threshold.

5. The method of claim 3 wherein the plurality of features further comprises:
the minimum diameter; and
the first component of the first color space, wherein the first component of the first color space is within a range of first components of the first color space including one or more of (1) a first first color space first component threshold, and (2) a second first color space first component threshold that is greater than the first first color space first component threshold.

6. The method of claim 5 wherein the algorithm criteria predicts malignancy when:
the maximum intensity is less than the first maximum intensity threshold;
the colocation is less than the colocation threshold;
the minimum diameter is greater than or equal to a minimum diameter threshold; and
the first component of the first color space is less than a first first color space first component threshold.

7. The method of claim 3 wherein the plurality of features further comprises the mean intensity.

8. The method of claim 7 wherein the algorithm criteria predicts malignancy when:
the maximum intensity is less than the first maximum intensity threshold;
the colocation is less than the colocation threshold;
the minimum diameter is greater than or equal to the minimum diameter threshold;
the first component of the first color space is greater than or equal to the first first color space first component threshold; and
the mean intensity is less than a mean intensity threshold.

9. The method of claim 3 wherein the algorithm predicts malignancy when:
the maximum intensity is less than the first maximum intensity threshold and greater than or equal to the third maximum intensity threshold;
the colocation is less than the colocation threshold;
the minimum diameter is greater than or equal to the minimum diameter threshold;
the first component of the first color space is greater than or equal to the first first color space first component threshold; and
the mean intensity is greater than or equal to the mean intensity threshold.

10. The method of claim 3 wherein the algorithm criteria predicts malignancy when:
the maximum intensity is less than the eighth maximum intensity threshold;
the colocation is less than the colocation threshold; and
the minimum diameter is less than the minimum diameter threshold.

11. The method of claim 3 wherein the plurality of features further comprises the first component of the second color space.

12. The method of claim 11 wherein the algorithm criteria predicts malignancy when:
the maximum intensity is less than the first maximum intensity threshold and greater than or equal to the fourth maximum intensity threshold;
the colocation is less than the colocation threshold;
the minimum diameter is less than the minimum diameter threshold and the intensity of the first component of the second color space is less than a second color space first component threshold.

13. The method of claim 3 wherein the plurality of features further comprises the centroid.

14. The method of claim 13 wherein the algorithm criteria predicts malignancy when:
the maximum intensity is less than the fourth maximum intensity threshold and greater than or equal to the seventh maximum intensity threshold;
the colocation is less than the colocation threshold;
the minimum diameter is less than the minimum diameter threshold;
the first component of the second color space is less than the second color space first component threshold; and
the centroid is greater than or equal to a centroid threshold.

15. The method of claim 3 wherein the plurality of features further comprises the first component of the third color space.

16. The method of claim 15 wherein the algorithm criteria predicts malignancy when:
the maximum intensity is less than the fourth maximum intensity threshold and greater than or equal to the seventh maximum intensity threshold;
the colocation is less than the colocation threshold;
the minimum diameter is less than the minimum diameter threshold;
the first component of the second color space is less than the second color space first component threshold;
the centroid is less than the centroid threshold; and
the first component of the third color space is less than a third color space first component threshold.

17. The method of claim 3 wherein the plurality of features further comprises the saturation.

18. The method of claim 17 wherein the algorithm criteria predicts malignancy when:
the maximum intensity is less than the second maximum intensity threshold and greater than or equal to the fifth maximum intensity threshold;
the colocation is less than the colocation threshold;
the minimum diameter is less than the minimum diameter threshold;
the first component of the second color space is greater than or equal to the second color space first component threshold; and
the saturation is greater than or equal to a saturation threshold.

19. The method of claim 3 wherein the algorithm criteria predicts malignancy when:
the maximum intensity is less than the sixth maximum intensity threshold and greater than or equal to the seventh maximum intensity threshold;
the colocation is less than the colocation threshold;
the minimum diameter is less than the minimum diameter threshold;
the first component of the first color space is less than a second first color space first component threshold that is greater than the than the first first color space first component threshold; and
the first component of the second color space is greater than or equal to the second color space first component threshold.

20. The method of claim 1 wherein processing the detected features includes processing the detected features by one or more processors executing instructions of a trained neural network defining the algorithm criteria.
